(19) United States
(12) Patent Application Publication
(10) Pub. No.: US 2011/0171105 A1
(43) Pub. Date: Jul. 14, 2011

(54) System and Process for Capturing, Concentrating, or Crystallizing a Target Compound from a Mixture

(76) Inventor: David Sevier, Surrey (GB)

(21) Appl. No.: 13/119,432

(22) PCT Filed: Sep. 16, 2009
(86) PCT No.: PCT/GB2009/051203

§ 371 Date: Mar. 16, 2011

(30) Foreign Application Priority Data
Sep. 17, 2008 (GB) 0817050.8
Nov. 12, 2008 (GB) 0820627.8
Feb. 6, 2009 (GB) 0901883.9
Jun. 25, 2009 (GB) 0910958.8

(51) Int. Cl. C01B 31/20; B01J 19/00; B01D 9/00; C01B 21/22; C01C 1/26; C01D 7/12; C01F 11/18; C07C 7/00

(52) U.S. Cl. 423/420; 422/255; 423/437.1; 422/245.1; 423/400; 423/421; 423/430; 423/438; 585/833

(57) ABSTRACT

The invention concerns a capture tank for capturing a captive target compound from a gaseous and/or vaporous mixture comprising at least the captive target compound and one other material, or for capturing, concentrating or crystallising a target compound from a liquid mixture or solution comprising the target compound and at least one other material, the capture tank comprising an enclosure having a top region, a bottom region and at least one side defining the enclosure, the enclosure being at least partly open in its top region in order to communicate in use of the capture tank with the gaseous and/or vaporous mixture and for permitting ingress of a gaseous and/or vaporous mixture into the enclosure; the enclosure communicating in its bottom region with a reservoir for receiving the captured captive target compound; having means associated with its at least one side and/or its bottom region for permitting egress from the enclosure of the gaseous and/or vaporous mixture in at least partially captive target compound-depleted form; and having means for sparging at least partially through the enclosure from top to bottom a liquid mixture or solution for entraining the gaseous and/or vaporous mixture in the enclosure and carrying the entrained gaseous and/or vaporous mixture towards the bottom region of the enclosure, and a process for its operation.

The present invention concerns a process and plant for capturing a captive target compound from a gaseous and/or vaporous mixture comprising at least the captive target compound and one other material or for recovering or concentrating a target compound from a liquid mixture or solution comprising at least the target compound and one other material. The invention has particular applicability in connection with environmental improvement, and may be used for example to remove greenhouse or pollutant gases from the atmosphere or pollutants from a waste stream. It may also be used to recover useful materials from waste streams or from other sources, and may be used for example to recover and/or concentrate for recovery pollutants from a waste stream or to effect crystallisation of the target compound.

This specification will emphasise the suitability of the invention to effect carbon capture—specifically carbon dioxide removal—from the atmosphere or from carbon dioxide enriched air or from waste streams comprising carbon dioxide in significant quantities, but it will be understood from the foregoing that the inventive process and plant may be utilised much more widely.

Currently the concentration of carbon dioxide in the atmosphere is rising and has been identified as being the principal greenhouse gas causing climate change. There is a great need to find a way to reduce or stop the rise of atmospheric carbon dioxide levels in order to manage climate change. Previous attempts by others to create viable methods of extracting carbon dioxide from the atmosphere have run into difficulties due to relatively high nervy use, high build costs and difficult economics. The processes outlined in this specification are aimed at specifically addressing these issues. The processes of the invention are also highly scalable as it will be necessary to carry out atmospheric carbon capture on a significant scale to address the problem of climate change.

Carbon dioxide is currently present at approximately 385 ppm in the air. This means that it is very diffuse and large quantities of air need to be mixed with an absorber to extract any meaningful amount of carbon dioxide. This is difficult to do conventionally at a meaningful rate and still use low energy. Wind has been considered for this but it is generally not continuous and this has the effect of decreasing the return on the capital build and increasing operational costs of the capture plant. Similarly, fans have a capital cost and require significant electricity to operate.

There is also a pressing environmental need to provide an improvement in currently available processes for the recovery of pollutants from waste streams and/or the concentration of such materials for subsequent recovery, and for effecting crystallisation of target compounds from solutions thereof.

The present invention seeks to address these difficulties.

According to the present invention there is provided a process for capturing, r concentrating or crystallising a target compound from a mixture comprising the target compound and at least one other material, the process comprising:
providing an enclosure having a top region, a bottom region and at least one side defining the enclosure, the enclosure:
   communicating in its top region with a gaseous and/or vaporous mixture for permitting ingress of the gaseous and/or vaporous mixture into the enclosure;
   communicating in its bottom region with a reservoir for receiving the captured or concentrated target compound;
   having means associated with its at least one side and/or its bottom region for permitting egress from the enclosure of the gaseous and/or vaporous mixture;
   having means for sparging at least partially through the enclosure from top to bottom a liquid mixture or solution, wherein the target compound is present in the gaseous and/or vaporous mixture and/or in the liquid mixture or solution;
sparging the liquid mixture or solution through the enclosure to create a downdraft of the gaseous and/or vaporous mixture through the enclosure;
   when the target compound is present in the gaseous and/or vaporous mixture providing as or in or in admixture with the liquid mixture or solution and/or in the reservoir an active agent having the capacity to interact with the captive target compound to render it captured in non-gaseous and non-vaporous form;
   when the target compound is present in the liquid mixture or solution at least partially evaporating the liquid mixture or solution in the downdraft to concentrate or crystallise the target compound;
collecting the captured, concentrated or crystallised target compound in the reservoir; and
venting the gaseous and/or vaporous mixture, optionally in at least partially captive target compound-depleted, from the enclosure through the at least one side and/or through the reservoir.

The invention has applicability both in connection with the capture of target compounds from gaseous and/or vaporous mixtures, and in connection with the capture of target compounds from liquid mixtures or solutions. In the former case the target compound is present in the gaseous and/or vaporous mixture at the start of the process. In the latter case the target compound is provided as or as part of or in combination with the liquid mixture or solution. It is of course possible in some cases for the target compound to be present in both the gaseous and/or vaporous mixture and in the liquid mixture or solution, or for a first target compound to be present in the gaseous and/or vaporous mixture and for a second target compound to be present in the liquid mixture or solution.

Thus, according to one aspect of the present invention there is provided a process for capturing a captive target compound from a gaseous and/or vaporous mixture comprising at least the captive target compound and one other material, the process comprising:
providing an enclosure having a top region, a bottom region and at least one side defining the enclosure, the enclosure:
   communicating in its top region with the gaseous and/or vaporous mixture for permitting ingress of the gaseous and/or vaporous mixture into the enclosure;
   communicating in its bottom region with a reservoir for receiving the captured captive target compound;
   having means associated with its at least one side and/or its bottom region for permitting egress from the enclosure of the gaseous and/or vaporous mixture in at least partially captive target compound-depleted form;
   having means for sparging at least partially through the enclosure from top to bottom an liquid mixture or solution for entraining the gaseous and/or vaporous mixture in the enclosure and carrying the entrained gaseous and/or vaporous mixture towards the bottom region of the enclosure;
sparging the liquid mixture or solution through the enclosure and entraining the gaseous and/or vaporous mixture therein so that the gaseous and/or vaporous mixture flows towards the bottom region of the enclosure;
providing as or in or in admixture with the liquid mixture or solution, and/or in the reservoir, an active agent having the capacity to interact with the captive target compound to render it captured in non-gaseous and non-vaporous form;
collecting the captured captive target compound in the reservoir; and
venting the gaseous and/or vaporous mixture in at least partially captive target compound-depleted form from the enclosure through the at least one side and/or through the reservoir.

The captive target compound in this case may be selected from any one or more known gaseous or vaporous pollutants, greenhouse gases, or other undesirable environmental components, and/or it may be selected from useful compounds which it may be desirable to capture and re-use for a useful purpose or to directly decompose. Non-limiting examples of captive target compounds include carbon dioxide, methane and nitrous oxide. Carbon dioxide is a preferred captive target compound.

The gaseous and/or vaporous mixture may be the atmosphere or may be for example a waste stream from an industrial plant or mine.

In another of its aspects the present invention provides a process for recovering or concentrating a target compound from a mixture comprising at least the target compound and one other material, the process comprising:
providing an enclosure having a top region, a bottom region and at least one side defining the enclosure, the enclosure:
   communicating in its top region with a gaseous and/or vaporous mixture for permitting ingress of the gaseous and/or vaporous mixture into the enclosure;
   communicating in its bottom region with a reservoir for receiving the recovered or concentrated target compound;
   having means associated with its at least one side and/or its bottom region for permitting egress from the enclosure of the gaseous and/or vaporous mixture;
   having means for sparging at least partially through the enclosure from top to bottom a liquid mixture or solution containing the target compound and at least one other material;
sparging the liquid mixture or solution through the enclosure to create a downdraft of the gaseous and/or vaporous mixture through the enclosure;
at least partially evaporating the liquid mixture or solution in the downdraft to concentrate or crystallise the target compound;
recovering the concentrated or crystallised target compound in the reservoir; and
venting the gaseous and/or vaporous mixture from the enclosure through the at least one side and/or through the reservoir.

The target compound in this case may be selected from any one or more known pollutants and/or it may be selected from useful compounds which it may be desirable to recover and re-use for a useful purpose. Non-limiting examples of target compounds include sodium phosphate hydrate or sodium sulphate hydrate. Hydrate salts are such as Glauber's salt (Na₂SO₄ 10H₂O) are particularly well suited to crystallizing using this method as the concentration of the dissolved salt (sodium sulphate) gradually increases beyond it's solubility point where upon crystallization occurs incorporating water. This has the effect of further reducing the available water to dissolve other sodium sulphate. The process evaporates water slowly enough to make large crystals grow but quickly enough to represent a viable method for producing large scale crystallization. The described crystallization process is applicable to non hydrated salts or compounds.

In the case where the process of the invention is used to crystallise a target compound, the at least one other material provided in admixture with the target compound may simply be a solvent or solvent mixture for the target compound. In this connection the word “mixture” in this specification expressly includes a solution comprising a mixture of solute and solvent.

The process of the invention facilitates with a relatively low energy requirement processes for concentrating dilute materials. Applications are numerous but include the concentration of pollutants in waste water to facilitate their eventual recovery and/or disposal, and the treatment of waste streams from the mining industry—for example to recover calcium sulphate or sodium phosphate therefrom.

In one process according to the invention the captive target compound may be captured by crystallisation. An example of such a compound would be sodium phosphate which can be supplied to the enclosure in the process of the invention in solution and crystallised in the downdraft, with sodium phosphate crystals being recovered from the process.

The enclosure is preferably defined by at least one side wall, which preferably has a circular cross section. However, substantially any configuration of side walls may be used to provide an enclosure having ovoid, polygonal or irregular cross section. The cross section need not be the same throughout the length of the enclosure, although it may be. The cross sectional area of the enclosure may be selected to suit the application, but will typically be at least about 1 m², or at least about 5 m², or at least about 10 m², or at least about 50 m², or at least about 100 m², or at least about 250 m², or at least about 500 m², for example.

The at least one side wall may be a solid wall constructed from any suitable material such as block, brick, panels—of metal or plastic for example, in the manner of a conventional chimney. However, it is also envisaged to use flexible materials—drapes, curtains and fabrics for example in the construction of the enclosure. A hollow cylinder of a suitable plastics material such as polypropylene or polyethylene for example would constitute a suitable arrangement for the enclosure.

It is also contemplated that the at least one side wall be constituted at least partially by a fluid material flowing continuously from top to bottom of the enclosure to generate a fluid curtain constituting the side wall. The fluid material may be a flowing solid such as a finely divided particulate material—sand for instance—but will preferably be a liquid, most preferably water or at least a water-based material.

The enclosure may be completely open at its top, thereby allowing maximum communication between the enclosure and the gaseous and/or vaporous mixture. However, in some instances it may be preferable partly to close the top of the enclosure—to filter debris or to direct downdraft flow, for example.

At its bottom the enclosure may also be completely open and in full communication with the reservoir. However, again in some instances it may be preferable partly to close the bottom of the enclosure, to filter debris or to direct recycle streams, for example.

The means for permitting egress of the gaseous and/or vaporous compound in at least partly captive target compound-depleted form may comprise one or more vents in the at least one side wall, preferably towards or in the bottom region of the enclosure. In the event that the at least one side wall is a continuously flowing side wall (a water curtain for example) then the at least one vent may be provided by deflecting the flow of fluid material in the at least one side wall, around a deflector plate or other kind or protuberance, for example.

In order to prevent the creation of negative pressure within the enclosure caused by wind blowing across the top of the enclosure, it is useful to install downward pointing louvers on the top of the enclosure to redirect the moving air downward into the enclosure. It is important to prevent the creation of negative pressure within the enclosure as this severely interferes with the downward flow of air.

Thus, there is also provided in accordance with the invention a process in accordance with the foregoing wherein means associated with the top region of the enclosure are provided for directing gaseous and/or vaporous mixture downwardly into the enclosure. Such means may comprise downwardly directed louvers, for example.

Preferably the sparging means is situated towards the top region of the enclosure. It may be situated at the top of the enclosure, but this may not be preferred in all cases—for example when the active agent is provided as or in admixture with the liquid mixture or solution and is a volatile compound which should not for preference be permitted to escape from the enclosure. The sparging means will generally be arranged to distribute the liquid mixture or solution across at least a major part of the cross-sectional area of the enclosure, such that the falling sparged liquid mixture or solution creates a downdraft in the enclosure.

An important feature of the process of the invention is connected with the capacity of the liquid mixture or solution to generate considerable downdraft in the enclosure and hence effect the movement of large volume of gaseous and/or vaporous mixture therethrough. This is particularly the case if the liquid mixture or solution has a vapour pressure such that at least partial evaporation of the liquid mixture or solution occurs in the enclosure. Evaporation of the liquid mixture or solution causes the temperature of the residual liquid mixture or solution in the enclosure to fall, and this in turn accelerates the downdraft. Consequently, in one preferred process according to the invention the liquid mixture or solution has a vapour pressure such that at least partial evaporation of the liquid mixture or solution occurs in the enclosure.

The liquid mixture or solution may be selected from any suitable material or mixture of materials, but will typically comprise water, which may be salt, waste or fresh.

It should be appreciated that when the active agent is provided in admixture with the liquid mixture or solution, such admixture need not necessarily occur prior to sparging of the liquid mixture or solution. For example, the active agent may if desired be sparged into the enclosure by second sparge means separate from the liquid mixture or solution sparge. For example, the process of the invention may use a dual sparge system in which a first salt water sparge entrains the gaseous and/or vaporous mixture which then passes on in the enclosure through a second fresh water sparge, in which the active agent is provided. In this manner, the entrainment of the gaseous and/or vaporous mixture is effected at least primarily by means of a salt water evaporate, and consequently relatively little or no evaporation of fresh water takes place. This may have advantages in localities where fresh water is in limited supply.

When the gaseous and/or vaporous mixture in at least partially captive target compound-depleted form is vented from the enclosure, it may be desirable to provide in the region of the vent a stripping mechanism for removing extraneous active agent, for example, from the vented stream. For example, the vented stream or at least part of it may be directed to pass through a flowing stripping medium, which may itself be a flowing water curtain for example. In this way, any extraneous active agent may be recovered from the vented stream by a stripping stream. However, it is emphasised that the stripping medium need not necessarily be water based, and could comprise non-volatile oil, for example

The active agent may be selected from materials which react chemically with or otherwise destroy the captive target compound—preferably to produce a non-gaseous and non-vaporous product—or which interact physically with the captive target compound, for example to adsorb the captive target compound on a surface of the active agent or to absorb or sequester the captive target compound within a matrix of the active agent. However, in this specification the word absorber will also be understood in context to refer to a chemically interactive material which has the effect of chemically absorbing the captive target compound in order for example to generate a new chemical entity, the captive target compound or a chemical constituent there of having been chemically absorbed by the active agent. For example, we shall refer herein to ammonia as a carbon absorber because it reacts chemically with carbon dioxide to generate ammonium bicarbonate.

In the case where the target capture compound is carbon dioxide, a preferred active agent is ammonia in combination with calcium sulphate or gypsum. In this case the chemical reactions which drive the process may be conveniently summarised as follows:

2NH₃ (gas)+2H₂O→2NH₄OH 1)

CaSO₄.2H₂O→CaSO₄ (dissolved)+2H₂O 2)

CaSO₄+CO₂+2NH₄OH→CaCO₃+H₂O+(NH₄)₂SO₄ 3)

In this manner it will be seen that carbon dioxide may be converted into captured form as calcium carbonate by interaction with the active agents in the form of ammonia and calcium sulphate. The gypsum may be dissolved or entrained as a suspension. The ammonia can be dissolved in the water or added as a gas. Higher capture rates occur if ammonia is added as a gas to the system. The use of gypsum is particularly advantageous because the gypsum may be supplied in the form of mining waste, which is often contaminated with calcium fluoride and radioactive materials. The process of the invention allows the selective dissolution of calcium sulphate from such waste streams and thereby effectively a means for recovering the calcium sulphate for further use.

Thus, in a preferred process in accordance with the invention the active agent is provided in the form of a gas and a direct gas-to-gas reaction occurs with the captive target compound to render the captive target compound captured. In one particularly preferred process in this connection the captive target compound is carbon dioxide and the active agent is ammonia. It is believed, although the process of the invention is not bound or limited by this theory that ammonia gas may react directly with carbon dioxide gas to form ammonium carbamate and ammonium bicarbonate. Both are unstable and subject to decomposition, but not sufficiently rapidly for the carbon dioxide not to be effectively captured. Both materials may proceed in an especially preferred process according to the invention to react with calcium sulphate to yield ammonium sulphate and calcium carbonate, thereby effecting long-term capture of the carbon dioxide captive target compound.

If desired, it is possible to vary reaction 3) to produce dry reaction products. This may be done by adding only one molecule of water to reaction 1 so that one molecule of ammonia and ammonium hydroxide are created. This is then fed into reaction 3 so that no water by-product is produced. This creates dry calcium carbonate (chalk) and ammonium sulphate.

Reactions 1-3 can be summarized by the equation below:

CaSO₄.2H₂O+CO₂+2NH₃→CaCO₃+(NH₄)₂SO₄+H₂O 4)

The process of the invention further envisages the subsequent regeneration of the captive target compound in a form suitable for downstream use. For example, when the captive target compound is carbon dioxide, and is captured in the form of chalk by reaction with ammonia and gypsum to generate chalk and ammonium sulphate the captive target compounds can be regenerated for use downstream. There are two routes to regenerate the ammonia and gypsum which will be further elucidated in the description of the preferred embodiments. The first is by thermal dissociation of ammonium sulphate to sulphuric acid and ammonia gas. The sulphuric acid is then reacted with the previously created chalk to produce a high pressure stream of carbon dioxide and gypsum. The reactions are:

(NH₄)₂SO₄→2NH₃+H₂SO₄ 5)

CaCO₃+H₂SO₄→CaSO₄+CO_{2↑}+H₂O 6)

The second route is by direct reaction of ammonium sulphate with chalk. At warm temperatures above 60° C., ammonium sulphate, chalk, and water react to form gypsum, ammonia gas, and carbon dioxide at high pressure. The reaction requires the constant input of heat to proceed forward. The reaction is:

(NH₄)₂SO₄+CaCO₃+H₂O→2NH_{3↑}+CaSO₄. 2H₂O+CO_{2↑} 7)

Reaction seven is highly advantageous because it can be powered by the waste heat created by such processes as electrical power generation. The described reactants outlined in the equations are calcium based. Any alkali metal including calcium is applicable.

Consequently, a preferred process in accordance with the invention includes at least one downstream step of regenerating the captive target compound, in this case carbon dioxide, for further use. Preferably, such downstream regeneration of carbon dioxide takes place by the reaction of calcium carbonate with ammonium sulphate, preferably driven by waste heat from an industrial process such as electrical power generation.

Also provided in accordance with the invention is a capture tank for capturing a captive target compound from a gaseous and/or vaporous mixture comprising at least the captive target compound and one other material, or for capturing, concentrating or crystallising a target compound from a liquid mixture or solution comprising the target compound and at least one other material, the capture tank comprising an enclosure having a top region, a bottom region and at least one side defining the enclosure, the enclosure being at least partly open in its top region in order to communicate in use of the capture tank with a gaseous and/or vaporous mixture and for permitting ingress of the gaseous and/or vaporous mixture into the enclosure; the enclosure communicating in its bottom region with a reservoir for receiving the captured captive target compound; having means associated with its at least one side and/or its bottom region for permitting egress from the enclosure of the gaseous and/or vaporous mixture in at least partially captive target compound-depleted form; and having means for sparging at least partially through the enclosure from top to bottom a liquid mixture or solution for entraining the gaseous and/or vaporous mixture in the enclosure and carrying the entrained gaseous and/or vaporous mixture towards the bottom region of the enclosure.

Also provided in accordance with the invention is a capture tank as herein before described constructed and arranged to operate the process of the invention as herein before described.

The invention will now be more particularly described with reference to a number of preferred aspects in connection with carbon capture, specifically carbon dioxide. It will be understood from the foregoing that other types of greenhouse gases or environmental or industrial pollutants or useful compounds may also be captured by means of the inventive process and plant, and the following description should be understood in that context.

It will be appreciated that, generally speaking, this invention overcomes the outlined problems of the prior art by inducing a flow of air in the enclosure, and by creating a reverse chimney effect through evaporative cooling (air is cooled by water evaporation in the case where the liquid mixture or solution is water) and/or by entrainment of gas by falling water droplets.

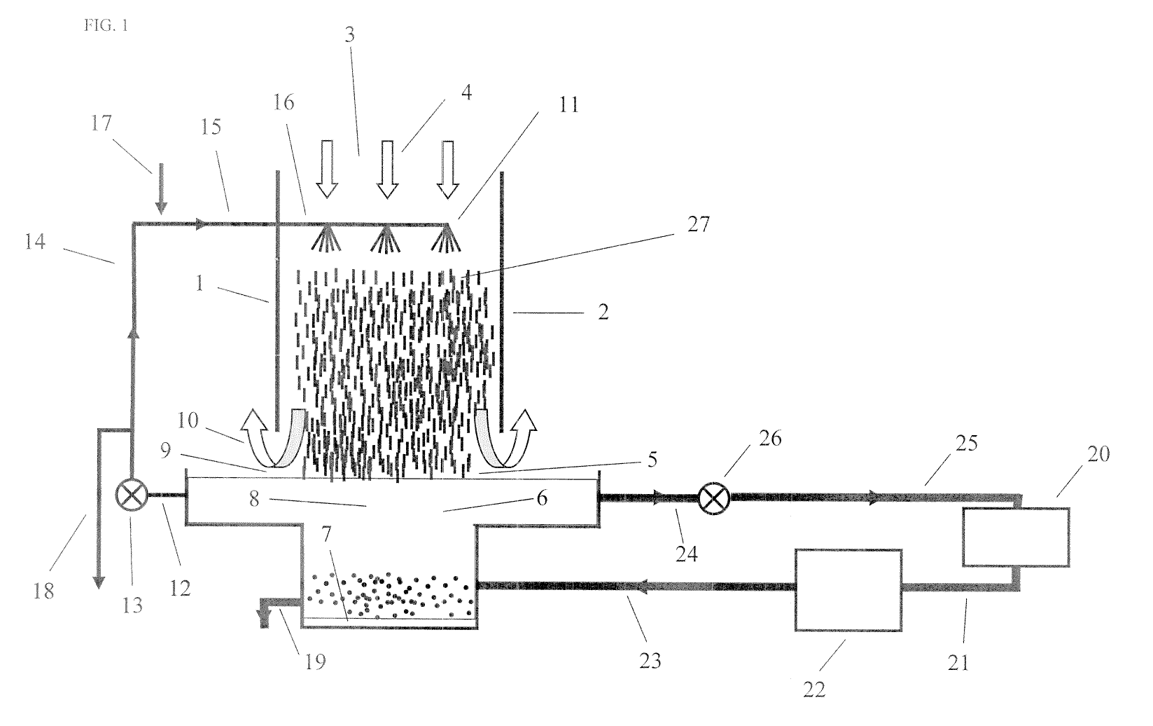

When water evaporates, it cools the non-evaporated water and the surrounding air. This effect is used by cooling towers and animals to disperse heat. Cooling towers are very effective evaporators of water because they mix amounts of high surface area water created by spraying fine mists or passing thin films of water over fill packs with large amounts of air. Cooling towers can produce cooling effects on the air and water passing though them of 10° C. of more. Cooling towers do not produce downward flows of air because they radiate an excess of heat such that the air entering the cooling tower is cooler than the air leaving the process. An air capture process that sprayed or passed water over fill packs would not experience a temperature gain but rather a temperature drop. If this was done in an open topped tank that had an opening at the bottom sides (see FIG. 1), then a movement of air would be created downward. This is because the air entering the top of the tank would be warmer than the cooler air leaving the tank at the bottom. The evaporation of the water, in addition to cooling the air through evaporation, would raise the humidity of the air leaving the tank. Humid air is heavier than non-humid air and sinks. Equally depending upon the size of the droplets of water sprayed, there is some transfer of downward momentum to the water from the air.

The downward flows of air generated by the evaporated of water can be large and can be generally calculated from the chimney equation. The equation does not take into account air density changes or water to air entrainment effects. The equation is:

$$Q = CA \sqrt{2 gh \frac{{T}_{i} - {T}_{o}}{{T}_{i}}}$$

where:
Q=stack effect draft/draught flow rate, m³/s
A=cross sectional area of air leaving tank, m³
C=discharge coefficient (usually taken to be from 0.65 to 0.70)
g=gravitational acceleration, 9.81 m/s²
h=height of the chimney, m
Tᵢ=air entering tank temperature, K
Tₒ=air leaving tank temperature, K

In essence, the enclosure used in the invention operates as a chimney in reverse with colder air at the bottom and warmer air at the top. An example is useful to explain the effect further:

For a capture tank that had a top cross sectional area of 400 m where
A=324 m³
C=065
g=9.81 m/s²
h=15 m
Tᵢ=298 K
Tₒ=295 K

The airflow rate is 362.5 m³/sec. The speed of the airflow rate through the top cross section is only 1.12 metres per second. The air contact time with active agent (for example an absorber) is 13.4 seconds.

In essence, large airflows may be generated in the enclosure but the speed of air is slow. This is ideal for air capture of the captive target compound (carbon dioxide for example) as the process limiting step is the slow rate of interaction between the gaseous captive target compound and the liquid or solid surface that contains the active agent, for example the slow rate of diffusion of carbon dioxide into an absorber that is dissolved in water. Generally, the rate of diffusion of the gas into the surface of the absorber is several orders of magnitude slower than the rate of reaction with between the target compound and the absorber(s). It is therefore the rate limiting step of the overall process. Faster air speeds reduce the length of time available for the carbon dioxide to diffuse into the water and then react with the carbon absorber(s). This tends to be counter productive and a balance needs to be struck such that sufficient air needs to pass through the air capture tank while providing sufficient time for the target gas to reach the absorber(s) and react.

It is generally less expensive in terms of capital build costs to create fine sprays of water for large volumes instead of filling a large void with fill packs. If water sprays are used, it is beneficial to use as fine a spray as practicable as this increases the surface area available for diffusion of carbon dioxide into the absorber. This has the effect of increasing the amount of carbon dioxide harvested from the air. If fill packs are used, packs that have a high surface area to pressure drop are the most beneficial. Equally, it is possible to create large surface areas using large open cell plastic foam. Such foam has the advantage of being compressible and compactable for shipping and being able to return to a low density material when allowed to expand. This greatly reduces transport costs. Produced products such as fill packs suffer from being highly voluminous by nature and have relatively high shipping costs. It is possible to combine the use of sprays, fill packs and/or open cell foams.

Accordingly, the invention also provides a capture tank for a captive target compound in accordance with the aforesaid description and statement of invention wherein the capture tank is provided with a fill material. Preferably the fill material has a high surface area to volume ratio. Preferably the fill material has an open cell structure. For example, open cell foams may used. Especially preferred are compressible open cell materials, which may be compressed to facilitate of transport and storage

Fine water sprays or mists will not settle out of the air before the air leaves the capture tank. To avoid the lost of absorber, it is necessary to have a water curtain of coarse spray to remove the entrained absorber or to pass the air through a drift eliminator.

As well as the providing improved methods for capturing atmospheric materials, the process of the invention may have a further benefit in connection with the generation of water vapour. The induced flow capture tower could be looked at as a way to evaporate large volumes of water for low energy. Possible applications include the use of such evaporate to concentrate dilute pollutants in waste water. If waste water (a useful humidity source) is used as the evaporation water source, very dilute pollutants are made significantly more concentrated and can then be removed.

Another ancillary benefit of the invention may lie in the productive use in carbon capture of waste gypsum created by mining (particularly phosphate mining) or gypsum recycling. The gypsum in phosphate mining waste stacks is currently considered useless as it is contaminated with naturally occurring radioactive minerals and calcium fluoride. The process of the invention can be operated to dissolve the gypsum but the radioactive minerals and the calcium fluoride are not soluble. This makes separation and clean up possible.

Another possible benefit of the inventive process lies in the provision of a possible source of supply of neutral carbon chalk for cement manufacture. Cement manufacture takes limestone or chalk and heats it to release the CO₂. The process of the invention could capture the CO₂ back from the air and turn it into chalk to feed back to the cement manufacturer. Approximately 60% of emissions from cement manufacture come from the decomposition of the calcium carbonate.

Another aspect of this invention concerns the use of a plural sparge system utilising both salt or waste water and fresh water sparges, the objective being to minimise usage of fresh water, particularly in those localities where supplies of fresh water may be limited. As will be apparent, air based carbon capture has the potential to evaporate very large amounts of water due to the huge amounts of air that need to be processed. Even small amounts of water evaporation relative to the air that passes through the process can lead to significant amounts of water make-up being required. Fresh water is a limited resource that is seeing increased pressure. The creation of an application that will further increase fresh water demand is likely to create use conflicts. It is possible to operate air based carbon capture using highly concentrated alkali solutions such that little or no evaporation takes place or to use selective ion plastic sheets but these processes suffer from a number of drawbacks such as high capital cost, alkali drift, and the need to operate fans to generate continuous air flow. The lack of self generating airflows is a significant problem because it imposes a sizable energy burden on the process of air based carbon capture and limits the ability to prevent alkali drift from the process. In general, it is easier to use dilute water based capture solutions as the evaporating water effects can be harnessed to induce air flows through the process. This saves energy and capital build cost. It is useful to have sprays located between the humidity source sprays (salt or waste water) and the capture side of the process to remove drift from the process that may contain absorbent and created salts. This has the advantage of not creating salt or chemical contamination within the carbon absorption side of the carbon capture process as the later separation of the contamination complicates the process. Contamination separation adds cost and increases the energy consumption of the process. The concentration of salts or contaminants in the humidification source water can be increased many times so that the solution leaving the process is of much smaller volume to that used to make up to the process

In one of its aspects, this invention therefore concerns the use of salt or waste water to create induced air flows and to limit fresh water evaporation from a carbon absorption process. This is achieved by initially passing the air through a fine spray of salt or waste water such that water evaporates and increases the humidity of the air. The cooler and high humidity air is then passed to the carbon capture process which can be based on fresh water. The high humidity air is either at or near saturation humidity and therefore will evaporate little or no water from the fresh water side of the process. This dual process can be optimized such that little water evaporates from the fresh water side of the process and is instead evaporated from the salt or waste water.

The evaporation of water is a function of a high surface to air ratio. It is therefore preferable to create fine water sprays as these will increase the evaporation of water. It is also possible to achieve the same effect using thin films of water such as would be created in cooling tower fill packs. Either sprays or fill packs will create a drift of salt or waste water that will contaminate the carbon absorption side of the process. This drift can be eliminated by adding a spray/thin fluid film on fill packs between the salt water spray and the carbon absorption side of the process. This will capture the contaminate drift. It will create a small amount of low contaminant drift (from the second spray) but this can be managed by controlling the concentration of contaminants such as salt in the second spray loop. It is also possible to add a further spray/thin fluid film on fill packs to further reduce the contaminant drift. It should be noted that the adding of extra steps in the process will increase the pressure drop across the overall process and will reduce the air flow/increase the energy that needs to be imparted to the fan that drives air through the process (if used).

One way to manage the contaminant concentration in the drift reducing step is to continuously transfer a proportion of the washing fluid if it is water based to the humidity source spray. This will consume a small but acceptable amount of fresh water.

The previously described evaporation process can be used to concentrate the carbon absorber such that highly concentrated solutions are produced. This is an advantage as more concentrated solutions generally require less energy to process. Equally, small volumes of liquid absorber generally requires less voluminous equipment which means that lower capital costs are required. It is equally useful for generating concentrated solutions of by-products from the carbon capture process.

One mode of operation of the process of the invention in this connection concerns the use of a reverse chimney that induces a large down draft of air by the evaporation of water which creates air cooling and increased air density. In this embodiment, air enters at the top of the chimney and is mixed with a spray of salt water such that the air becomes saturated with humidity. The cold denser air falls and passes through a spray of fresh water that removes the high salt drift from the salt water sprays. The salinity of the wash sprays are controlled by continuously adding a proportion of the wash water to the salt sprays and making up the wash spray with fresh water. The air then passes to the carbon absorbing side of the process where carbon dioxide is removed from the air. The carbon absorber system may or may not be liquid and may or may not be based upon fresh water solutions of absorber. The air then leaves the carbon absorbing side and may or may not pass through a drift reduction water spray before leaving at the bottom of the chimney. The process is generally designed to mainly evaporate water from the salt water side of the process and not the carbon absorbing side. The process is configured to minimize the mixing of the different water sprays. There are a many ways to make this happen that will be apparent to those who are experienced in this work. A simple illustration of one such solution is a straight vertical tube that is open at the top and the bottom. Air enters the top of the tube and passes through the salt water spray and gains humidity. Near the top of the tube is a “floor” that salt water sprays fall into. The air is allowed to fall out of the enclosed and bulged sides of the tube located above the salt water spray floor that located within the tube. Within the bulged sides, fresh water is sprayed to eliminate the high salt drift that is mixed with the air. The air continues to fall and enters the carbon absorbing part of the process that is located below the salt water spray floor. The air falls down the tube through the capture process and then leaves at the bottom of the tube. Lips and fluid barriers are installed in the appropriate places to prevent the flowing of the various liquids to other parts of the process. The fluids may be continuously reused.

The efforts to develop and implement carbon capture from waste streams and the air have been severely hampered by the difficulties of regenerating carbon dioxide absorbers at low temperatures. Typically the temperatures required to regenerate the absorber systems are hundreds of degrees Celsius. This imposes significant cost and energy restrains on the carbon capture process.

Another advantage of the invention is that the captured products of the process may if desired be regenerated for downstream use, and that such regeneration may be effected at relatively low temperatures, such that the by-products of the absorption process are regenerated below 100° C. The chemical reactions for the regenerative production of carbon dioxide in circumstances where the active agent is a combination of ammonia and gypsum have been summarised previously.

The regeneration reaction (reaction 7) requires the input of heat. The reaction proceeds forward as heat is inputted into the system. The reaction occurs at and below the boiling point of water. Excess water does not hinder the reaction and is generally helpful. Generally, the reaction speed is governed by the rate of heat input into the system if fine powdered chalk is used. Warmer temperatures generally increase the rate of reaction. Gypsum is created by the reaction and is of low solubility and precipitates out. Ammonia gases out of the system with the released carbon dioxide. It is important to keep the released gases warm so that ammonium bicarbonate is not formed. If the gases are kept above the temperature which ammonium and carbon dioxide react to form stable ammonium bicarbonate and ammonium carbamate, the gases can be passed through water curtains and the ammonia separated from the carbon dioxide. Ammonia is highly soluble in water and carbon dioxide is generally of low water solubility if the pressure is kept low. This allows for straight forward separation. The created ammonium hydroxide is recycled back to reaction 3 to fix more carbon dioxide.

The process may be further improved by recirculating carbon dioxide back through the regenerative system where reaction 7 is occurring in order to strip ammonia from the slurry and improve the reaction rate.

To capture CO₂ from high concentration CO₂ gas streams, it is necessary to spray water slurries of gypsum and ammonia as the low solubility of calcium sulphate in water becomes a problem. This produces a mixture of gypsum and chalk. This mixture (or just chalk) and the created ammonium sulphate dissolved in water can be heated to create ammonia vapour, carbon dioxide and gypsum.

It is possible to use waste process heat from a source such as an electricity power plant to provide the heat required to make reaction 7 proceed forward. Reaction 5 and 7 produces ammonia and gypsum which can be recycled to produce circular reaction cycles such as the ones outlined in cycle one and two. These cycles continuously recycle the reactants with the exception of carbon dioxide. The net result is a chemical system capable of concentrating dilute carbon dioxide into a pure carbon dioxide stream. The ability to use plentiful low grade waste heat as the heat source to regenerate the sorbent system in cycle 1 is highly advantageous.

Cycle 1 is:

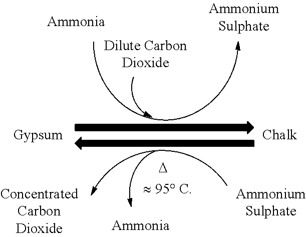

The chemical reactions are:

NH₃+H₂O→NH₄OH a)

CaSO₄.2H₂O→CaSO₄ (dissolved)+2H₂O b)

CaSO₄+CO₂+2NH₄OH→CaSO₃+H₂O+(NH₄)₂SO₄ c)

(NH₄)₂SO₄+CaCO₃+H₂O→2NH_{3↑}+CaSO₄.2H₂O+CO₂ d)

Low temperature heat is applied to make reaction d proceed forward.

Cycle 2 is:

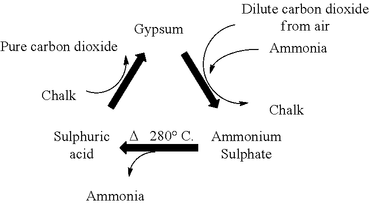

The chemical reactions are:

NH₃+H₂O→NH₄OH a)

CaSO₄.2H₂O→CaSO₄ (dissolved)+2H₂O b)

CaSO₄+CO₂+2NH₄OH→CaCO₃+H₂O+(NH₄)₂SO₄ c)

(NH₄)₂SO₄→2NH₃+H₂SO₄ e)

CaCO₃+H₂SO₄→CaSO₄+CO_{2↑}+H₂O f)

Reaction e proceeds forward at approximately 280° C. depending upon conditions.

The invention will now be more particularly described with reference to the drawings in which:

FIG. 1 which shows in schematic form a carbon capture plant in accordance with the invention;

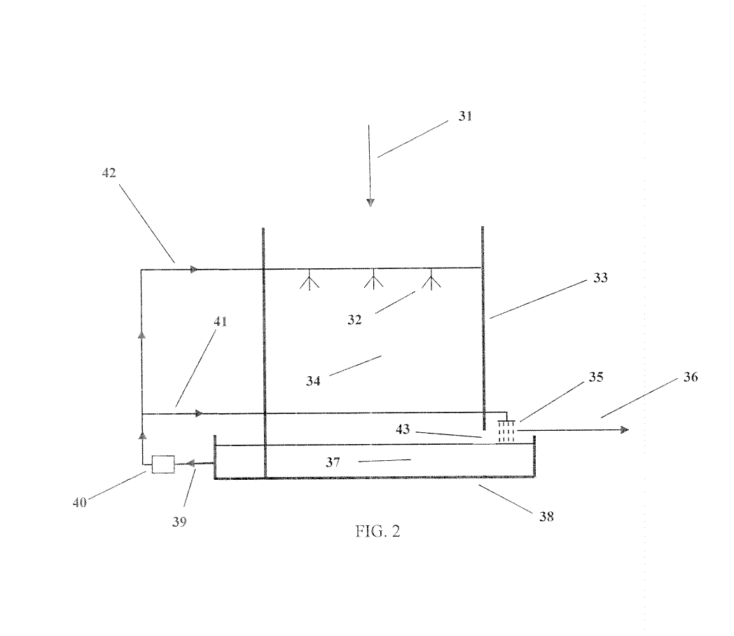

FIG. 2 shows an alternative arrangement of such a plant.

Referring to FIG. 1, there is shown enclosure 1 defined by cylindrical side wall 2 which in this embodiment is a solid wall built from blocks or other suitable material. Top region 3 of enclosure 1 is open to the atmosphere, the purpose of the plant depicted in FIG. 1 being to capture carbon dioxide therefrom. Arrows 4 indicate the passage into enclosure 1 of atmospheric air in operation of the plant.

Bottom region 5 of enclosure 1 communicates with reservoir 6 which in this embodiments acts both as the collection means for captured carbon (shown in schematic form as settled chalk at 7) and as a storage container for ammonium sulphate solution 8 which in this embodiment forms, together with dissolved calcium sulphate, one of the active agents for the process.

Enclosure 1 is provided at its bottom end with vents 9 which permit egress of CO₂-depleted air as indicated by arrows 10 in operation of the plant.

Sparge 11 situated towards the top of enclosure 1 and is fed with a mixture of calcium sulphate solution and ammonia, the calcium sulphate solution being supplied from reservoir 6 through line 12, recycle pump 13, and lines 14, 15 and 16. Ammonia is supplied to the system in line 17, and line 18 is a bleed line for withdrawing ammonium sulphate solution from the recycle stream to prevent its buildup in the system.

Chalk may be periodically or continuously extracted in line 19, whilst calcium sulphate is continuously or periodically supplied to the system in gypsum mix tank 20, before flowing on in line 21 to gypsum settling tank 22 where any insoluble gypsum salt is allowed to settle, and from where calcium sulphate solution flows on in line 23 to reservoir 6. The calcium sulphate content of reservoir 6 is maintained by recycle through lines 24, 25, mix tank 20, line 21, settling tank 22 and line 23, the recycle being driven by recycle pump 26.

In operation of the plant, an active agent flow consisting in this case of calcium sulphate solution and ammonia is caused by the operation of recycle pump 13 to flow into sparge 11 and fall through enclosure 1 as a falling fine absorbent spray 27 which entrains air from the top region of enclosure 1 and causes a downward flow of air therein. The sorbent spray evaporates water as it falls and raises the air density, causing the air in the region of evaporation to fall and enhance the downdraft effect in enclosure 1, further enhancement of this effect being caused by the cooling effect on the air of water evaporation.

As the active agent spray and the entrained atmospheric air fall through the enclosure, dissolved calcium sulphate combines with ammonia and carbon dioxide from the air to form ammonium sulphate and chalk according to the previously described and discussed equation 3.

The recovered chalk settles in solid form at the bottom of reservoir 6, whilst CO₂-depleted air is vented from enclosure 1 through vents 9, as indicated in the Figure. Ammonium sulphate solution is recovered in reservoir 6, and is bled from the system in line 18 to prevent its build-up.

Not shown in FIG. 1, but preferably present, is at least one drift eliminator or water curtain running down or near side wall 2 for stripping removal of any volatile substances such as extraneous ammonia for example present in the vented stream or particulate drift from the vented stream.

Consequently, it will be seen that in operation the plant of FIG. 1 provides an effective means for large scale removal from the atmosphere of carbon dioxide, and that major environmental benefits may be realised by the inventive process and plant.

Referring to FIG. 2, there is shown an induced draft carbon capture tank. Reference numeral 31 indicates the incoming air containing carbon dioxide. Water/absorber spray heads 32 produce a fine mist of absorber and water. Reference numeral 33 indicates the side wall of the carbon capture tank, and reference numeral 34 the falling mixture of fine mist water, absorber and air. Reference numeral 35 indicates the coarse spray of absorber/water to remove excess drift and mist from the air leaving the carbon capture tank through opening 43. Reference numeral 36 indicates carbon dioxide depleted air that has left the carbon capture tank. The humidity of air 36 is higher than the air 31 which entered the carbon capture tank due to water evaporation from the water absorber spray heads 32. Reference numeral 37 indicates the water/absorber mixture that has fallen from the carbon capture device, and 38 is the sump receiving the water/absorber mixture. Reference numeral 39 indicates the water/absorber leaving sump 38 going to recirculation pump 40 which pumps the water/absorber to line 41 delivering the water/absorber to spray head 35 of the water curtain and to line 42 delivering water/absorber to spray heads 32.

The outline air capture tank would generally have the format as outlined in FIG. 1 where air is drawn into the open top of the tank, mixes with fine water spray. The water spray evaporates water into the passing air and cools both the air and the water. The absorber within the water spray reacts/absorbs carbon dioxide from the air. The water falls to the bottom of the tank where it is collected and recirculated back to the spray heads. The cool dense air leaves the tank through the bottom sides of the tank where it passes through a water curtain to remove entrained water/absorber drift. The water curtain can use water that contains absorber (shown) to remove drift or it can use fresh water (not shown) to remove drift.

The outlined capture process will work with any absorber that can be dissolved or be entrained in water. The process can also be used with volatile absorbers such as ammonia. Ammonia used as a carbon dioxide absorber has the following advantages:
Low cost and widely available
Ammonia can be removed from water solutions by air stripping. Equally, ammonia can be removed from air by water curtains. Ammonia solubility is highly temperature dependent and allows good possibilities for manipulation of solubility properties.
Ammonia can be biodegraded by the environment.

If volatile carbon absorbers such as ammonia are used, then it is necessary to manage the vapour pressure issues of the absorber and created absorber and target compound leaving the carbon capture tank. This can be managed in two ways. The first is to use of water curtains that remove the ammonia vapour by taking advantage of the high solubility of ammonia gas in water. Generally, two separate water curtains to remove the ammonia vapours are needed. More or less water curtains are required depending upon the particular dynamic of the process being run in the carbon capture tank. In this way, ammonia vapour only remains within the tank and does not leave the process. Alternatively, ammonia can be added to the process such that all the added ammonia reacts to form ammonium sulphate which essentially has no ammonia vapour pressure. In this way, the need for extra water curtains is removed and no vapour is lost from the system.

Over time in operation of the process where water curtains are used, the water curtains' ammonia concentration rises such that the curtain will not remove sufficient ammonia. To control this, some of the water being used for the curtain must be removed and fresh water added. In the case of two or more water curtains to remove the ammonia vapour, fresh water is added to the outer water curtain. Water is removed from the outer curtain and added to the inner water curtain, and water is removed from the inner curtain and added to the general absorber solution circulating in carbon absorber tank to make up water evaporation losses. A gradient of ammonia concentration in the water curtains verses the air counter current is established to maximize the removal of ammonia from the air. The water losses from the carbon absorber tank process are significant but not enough to support the necessary refresh rate required to prevent excessive accumulation of ammonia within the water curtains. It is therefore necessary to regenerate some of the curtain water to control ammonia concentration. This is done by first heating the water so that the vapour pressure of ammonia is greatly increased and then air stripping the mixture to reduce the ammonia concentration. The regenerated water is then cooled and returned back to the water curtain. The use of counter current heat exchangers reduces the amount of heating and cooling required of the curtain water during the regeneration process. The air that is used to strip the ammonia out of the curtain water is passed to the top of the main carbon capture tank where it makes up a small fraction of the total air passing through the carbon capture tank.

The induced draft capture process can used to create useful by-products to supplement the economics of operating and building the carbon capture process. The ammonium sulphate cycle is particularly advantageous for this as it can be harnessed to produce a range of useful products. It can also be used to create a very high pressure stream of carbon dioxide such that further compression is generally avoided or greatly reduced prior to other use or disposal. This is advantageous in terms of reduced equipment and energy costs.

Reaction 3 occurs in the capture tank. It is not necessary to run the full reaction cycle. It is possible to use the reactions to run an open process to generate just ammonium sulphate, chalk, sulphuric acid, fine particle gypsum or a high pressure stream of carbon dioxide. Ammonium sulphate decomposes at 280° C. (reaction 5) which is below the boiling point of sulphuric acid. This means that it is fairly easy to separate the ammonia, which becomes gaseous, from the liquid sulphuric acid. If salt water is used as make-up to a process running the ammonium sulphate cycle, salt can be separated during the ammonium sulphate decomposition. Salt has virtually no solubility in anhydrous sulphuric acid and can therefore be simply strained out.

A good source of water to operate the ammonium sulphate air capture cycle with is to use wastewater from phosphate rock mining and processing. This water tends to contain a high level of dissolved calcium sulphate that is produced during the phosphate rock refining. As calcium sulphate is consumed in the ammonium sulphate cycle, this water is highly useful. Other wastewater sources are likely to have similarly advantages.

Equally useful is the ease with which the starting ingredients and created products from reaction 4 can be separated. Gypsum is sparingly soluble at approximately 2.8 g/litre. Chalk has very low solubility and precipitates easily. Ammonium sulphate is very soluble. This means that it is possible to create a process where all the dissolved gypsum reacts and forms ammonium sulphate and chalk. The created chalk simply precipitates out and leaves a solution of ammonium sulphate. If reactions 1, 2, and 3 are cyclically repeated, the result is a high concentration solution of ammonium sulphate.

A useful modification of the ammonium sulphate cycle is to use waste gypsum created by phosphate mining and refining, capture carbon dioxide from the air and create ammonium sulphate and chalk. The ammonium sulphate is decomposed with heat and pure sulphuric acid is created which is used as part of the phosphate mining and refining process. Phosphate rock is reacted with sulphuric acid to produce phosphoric acid and gypsum. The ammonia is returned back to the carbon capture process. The process has much to recommend itself. It consumes problematic waste products from the mining (wastewater and gypsum), eliminates the need to purchase sulphuric acid (phosphate mining and production uses nearly half of the world's production of sulphuric acid) and, sequesters carbon dioxide as highly stable chalk. The precipitated chalk can be used for a number of purposes such as paper making but is particularly helpful for stabilizing and buffering the acidic run off from the waste gypsum piles that create local environmental problems. Potentially, the described modified ammonium sulphate cycle can significantly reduce the environmental damage of phosphate rock mining and processing.

Reactions cycle 1d and cycle 2f as part of the ammonium sulphate cycle are very useful as they generate very high pressure carbon dioxide. Reaction cycle 2f is a driven reaction that tends to faster reaction rates as the pressure rises. This makes these reactions well suited for creating high pressure carbon dioxide gas. Generally, the need for further compression of the gas is eliminated. This is highly advantageous as compression equipment is a significant added cost and relatively energy intensive to operate.

It is also possible to operate the ammonium sulphate process such that carbon capture proceeds in the carbon capture tank in accordance with equation 3.

It is generally advantageous to have a separate pump operating to dissolve the gypsum and one to pump water and absorber into the carbon capture tank. The need for two pumps is due to the modest solubility of the gypsum. Significantly, more water needs to be circulated to dissolve the gypsum than needs to be pumped and sprayed into the tank to induce the airflow. It is therefore more efficient to separately circulate water to dissolve the gypsum without pumping it up hill and to only pump up hill the amount of water and absorber necessary to induce the down draft. In this way, both parts of the process can be optimized.

It is also generally advantageous to locate the chalk settling tank below the carbon capture tank. This arrangement means that less vapour containment is required as both the carbon capture tank and settling tank below share the same air space. Equally, this arrangement uses less land. This arrangement also avoids the need to create a separate sump area below the carbon capture tank.

Passing wind can disrupt the downward airflow through the tank if insufficient distance is not allowed for above the tank. It is therefore useful to allow a distance above the water sprays such that passing wind does not entrain the sprayed water absorber and remove it from the capture tank.

The described induced draft capture process can be used to capture gases other than carbon dioxide provided the correct absorber/reactant is used. Gases such as nitrous oxide or methane can be captured using this process. The use of the induced draft capture process to capture other gases from atmosphere is specifically contemplated herein.

Nitrous oxide can be captured and destroyed by reaction with dissolved sodium thiosulphate under alkaline conditions for example. The capture process like the process outlined for carbon capture is generally governed by the rate of diffusion into the water droplets. The process has the advantage that because the concentration of nitrous oxide in the air is low at only hundreds of parts per billion, only small amounts of sodium thiosulphate are required. Equally, the amount of created destruction products are small and can generally be disposed of without significant or any processing. Nitrous oxide while at low levels in the air is nearly three hundred times more potent a greenhouse gas than carbon dioxide. Large effects can therefore be gained by removing and destroying modest quantities of nitrous oxide.

As indicated hereinabove, it is possible to create induced draft carbon capture without permanent sidewalls. This modification reduces the build cost of the capture process. Walls are necessary to create the reverse chimney effect that draws air through the water spray. The walls do not have to be permanent walls and can instead be created by falling sheets or tight sprays of water. Some water fountains do this and create continuous falling curtains of water. This will create the same chimney effects as permanent walls. Openings at the bottom of the falling curtains of water are still required so that air can escape. This can be accomplished by simple defection to create an opening in the water curtain. An improvement is to have the water fall onto coarse open cell foam. This will provide a suitable air exit and traps the fine spray drift.

Using temporary walls of water will mean that more water needs to be pumped but the overall cost for water pumping is quite low and is a small fraction of the cost of capital. Wind can disrupt the water walls and the airflow through the top of the spray tank but it generally replaces downward airflow with sideways airflow. The overall airflow is largely unchanged up to an upper threshold of wind speed that completely disrupts the induced draft.

The described induced downward air draft using temporary water walls can be created by having a water discharge ring that surrounds the spray head array to create the water walls. The height of the ring is several metres higher than the spray head array to avoid wind disruption effects. The water and absorber spray heads are mounted on poles. Depending upon the configuration, it can be advantageous to bind the poles to one another to create a more resilient structure. It is necessary to surround the induced air draft carbon capture using water walls with an impermeable membrane on the ground to catch excess water drift. To improve the economics of this, it is useful to cluster a number of capture units together and surround them with a common membrane. Vapour-less or low vapour pressure absorbers are required for use with a process that uses temporary water walls.

Induced draft using temporary water walls will evaporate more water than a process that uses permanent walls. In essence, induced draft using water walls is optimized for water evaporation. This has application beyond carbon capture and can be used for positive weather modification. Specifically if the aforementioned induced draft with temporary water walls is used to evaporate seawater with no consideration made to capture carbon dioxide or prevent drift, cheap highly efficient water evaporation devices can be created. The devices circulate seawater from below the structure. In essence, a ring of spray heads on poles creates the temporary water walls. The ring surrounds an array of spray heads on poles that produce the evaporating water spray that causes the downward draft of air. The structure has the advantage of being of low cost to build and construct, and of offering low resistance to the damaging action of waves.

It is useful to calculate just how much water these structures could evaporate to show the very large amounts of water evaporated to power consumed. In this example the air entering the water evaporation device has 70% humidity at 25° C. which contains 16.1 g of water per m³ of air. The air leaving the water evaporation device has 90% humidity at 25° C. that contains 20.7 g of water per m³ of air. The density of air=1200 g/M³. The specific heat of air=1.012 J/g/° C. For ease of calculation, it is assume that the density and the specific heat of the air do not change as humidity increases. The energy to evaporate 1 gram of water at 25° C.=2.258 kJ/g. The device has a spray area of 169 m² and has walls that are 7 metres high and which spread the water out in the lower two metres so that an opening of 100 m² is created,

The device adds 4.6 g of water per cubic metre of air passing through the process. The evaporation of water also reduces the temperature of the air by 8.5° C. If the water evaporation devices had a discharge coefficient of 0.65, only 11,086 units would be required to evaporate a cubic kilometre of water over a year. This volume of water is approximately equal to covering the state of Israel with 5 cm of rain. 11,086 units would also have the effect of cooling approximately 607 cubic kilometres of air 8.5° C. per day. The predicted electricity consumption for the 11,086 devices with solid walls would be slightly over 1 million KWH per 24 hour day. This is a fraction of the power requirement to carry out sea water desalination using traditional means to produce 1 km³of freshwater.

The described water evaporation effects will be greater for lower humidity warm air.

Thus, it can be seen that it is practicable to use the described seawater evaporation devices to modify local climate, in addition to the benefits of carbon capture.

The induced flow spray tower is good at creating mass crystallization of solutions that are dissolved in the water that is sprayed within the tower. The process uses very low energy and can create large crystals. If the tower is used for crystallization, seed crystallization points need to be placed in the tower for crystals to grow on. This can be rods or strings. The crystals grow on the rods or strings and can be harvested with ease and returned to the tower for reuse.

The invention will now be more particularly illustrated by an example in which the plant of FIG. 1 is deployed in carbon capture using an active agent comprising gypsum and ammonia.

### EXAMPLE

In a 1.44 M² induced flow reverse chimney created by spraying fine droplets of water solution, air was drawn into the unit, schematically shown in FIG. 2. The water had dissolved within it calcium sulphate from gypsum and ammonia which was added to the water. Only enough ammonia was added such that all the ammonia reacts to form ammonium sulphate which has essentially no vapour pressure. In this way, ammonia vapour leaving the process was avoided. Drift eliminators in the form of fine nylon mesh curtains were used to trap spray drift from the tower. The tower was six metres high and stood 1 metre off the ground and contained eight medium fine water spray heads with a fluid discharge rate of 12.6 litres per minute. Water was discharged from the recirculation pump at the bottom of the tower at a pressure of 40 psi. The piping from the pump to the spray heads had a diameter of 22 mm. The chimney was fitted with downward pointing louvers to prevent passing wind disrupting the airflows. Air flow speeds of 0.9 to 1.0 metres per second were observed. A minimum of four degrees temperature drop in air temperature was seen at the bottom of the tower. Both the temperature drop and the air flow varied slightly with ambient conditions such as passing winds, air humidity and temperature but were generally consistent. The process captured carbon dioxide from the air at the rate of 8.5 kg per day. Significant amounts of chalk and ammonium sulphate were created. The process pH was maintained above 7.0 with a small excess of ammonia but this could also have been done with a non-reactive base or a buffer.

The capture rate was generally governed by the total surface area of the drops so the finer the sprays, the better the capture rate. The general reactions that occur are in accordance with equations 1) to 3) which have been previously described.

The chalk that was produced was made up of very fine particles and has lots of uses. Ideally it is best to dissolve the gypsum away from the created chalk so that pure chalk precipitates at the bottom of the tower sump. It is important to avoid delivering entrained gypsum particles to the spray heads to avoid clogging if these are used to create the falling water.

Fresh water was used within the tower. Significant evaporation occurs unless a humidity source is provided for the air that passes through the process. If waste or salt water is sprayed (fine sprays) prior to the air entering the process, the air can be made saturated with humidity and virtually all the water losses can come from waste or salt water. Little water is therefore lost from the fresh water sourced solution that recirculates in the main tower which captures the CO₂. Alternatively, salt or waste water can be used within the tower but the created ammonium sulphate will be mixed with sodium chloride (if salt water is used). No separation of the two salts is required if the process is being operated to just capture CO2. The presence of sodium chloride has no adverse effect on reaction seven.

The previously described 1.44 M² capture tower was used to crystallize sodium sulphate hydrate in the presence of dissolved gypsum under alkaline conditions (pH of 10.8 to 11.4). The pH conditions were created by initial sodium hydroxide addition. The dissolved sulphate level was not determined prior to the start of crystallization but found to be approximately 69,000 ppm sulphate as SO₄ at the point of crystallization. The system volume was approximately 200 litres. No make up water was added to the system. In approximately 24 hours, mass crystallization occurred, creating large crystal masses. Some individual crystals were several centimetres across. The tower, sump and drift curtains were coated in a significant weight of large crystals. The approximate energy inputted to create the crystallization was calculated to be approximately 1.4 kilowatt hours.

It will be apparent to those who are experienced in the technology that there are further variations of the induced draft through evaporation, gas capture processes and production of high-pressure carbon dioxide that are described here.

What is claimed is:

1. A process for capturing concentrating or crystallising a target compound from a mixture comprising the target compound and at least one other material the process comprising:
a. providing an enclosure having a top region, a bottom region and at least one side defining the enclosure, the enclosure:
i. communicating in its top region with a gaseous and/or vaporous mixture for permitting ingress of the gaseous and/or vaporous mixture into the enclosure;
ii. communicating in its bottom region with a reservoir for receiving the captured or concentrated target compound;
iii. an opening in at least one side and/or a bottom region of the enclosure for permitting egress from the enclosure of the gaseous and/or vaporous mixture;
iv. a sparging device for sparging a liquid mixture or solution at least partially through the enclosure from top to bottom, wherein the target compound is present in the gaseous and/or vaporous mixture and/or in the liquid mixture or solution;
b. sparging the liquid mixture or solution through the enclosure to create a downdraft of the gaseous and/or vaporous mixture through the enclosure;
c. when the target compound is present in the gaseous and/or vaporous mixture, providing as or in or in admixture with the liquid mixture or solution and/or in the reservoir an active agent having the capacity to interact with the captive target compound to render it captured or destroyed in non-gaseous and non-vaporous form;
d. when the target compound is present in the liquid mixture or solution, at least partially evaporating the liquid mixture or solution in the downdraft to concentrate or crystallise the target compound;
e. collecting the captured, concentrated or crystallised target compound in the reservoir; and
f. venting the gaseous and/or vaporous mixture, optionally in at least partially captive target compound-depleted, from the enclosure through the at least one side and/or through the reservoir.

2. The process according to claim 1, wherein the process comprises for capturing a captive target compound from a gaseous and/or vaporous mixture comprising at least the captive target compound and one other material and wherein the gaseous and/or vaporous mixture vented in the venting step is in at least partially captive target compound-depleted form.

3. The process according to claim 2 wherein the captive target compound is selected from any one or more known gaseous or vaporous pollutants, greenhouse gases, or other undesirable environmental components, and/or from useful compounds desired for capture and re-use for a useful purpose and wherein the gaseous and/or vaporous mixture comprises the atmosphere or a waste stream from an industrial plant.

4. The process according to claim 3 wherein the captive target compound is selected from carbon dioxide, methane and nitrous oxide.

5. (canceled)

6. (canceled)

7. (canceled)

8. The process according to claim 2 wherein a dual sparge system is provided in which a first salt or waste water sparge entrains the gaseous and/or vaporous mixture which then passes on in the enclosure through a second fresh water sparge, in which the active agent is provided, wherein the active agent is selected from materials which react chemically with the captive target compound or which interact physically with the captive target compound.

9. (canceled)

10. The process according to claim 8 wherein the product of the chemical or physical interaction between the active agent and the captive target compound is non-gaseous and non-vaporous and wherein the active agent is provided in the form of a gas and a direct gas-to-gas reaction occurs with the captive target compound to render the captive target compound captured.

11. (canceled)

12. The process according to claim 3 wherein the captive target compound is carbon dioxide and the active agent is ammonia or ammonia in combination with an alkali metal or alkaline earth metal sulphate or calcium sulphate hydrate.

13. (canceled)

14. The process according to claim 12 wherein the alkaline earth metal sulphate is calcium sulphate provided in the form of a waste stream from a mine and is contaminated with at least one undesirable material, wherein the process allows the recovery of calcium sulphate from the said waste stream by selective reaction with the target compound.

15. (canceled)

16. The process according to claim 12 wherein the ammonia active agent is provided as a gas which reacts directly with carbon dioxide gas to form ammonium carbamate and ammonium bicarbonate.

17. The process according to claim 16 wherein ammonium carbamate and ammonium bicarbonate react with the alkali metal or alkaline earth metal sulphate to yield ammonium sulphate and alkali metal or alkaline earth metal carbonate.

18. The process according to claim 2 wherein the captive target compound is regenerated by further reaction wherein further reaction is with a by-product of the interaction between and active agent and the captive target compound and wherein the captive target compound is carbon dioxide and regeneration of carbon dioxide takes place by the reaction of calcium carbonate with ammonium sulphate or any alkali metal sulphate.

19. (canceled)

20. (canceled)

21. The process according to claim 18 wherein the regeneration reaction is driven by waste heat from a power plant.

22. The process according to claim 18 wherein the regenerated captive target compound is contained and pressurized for downstream use and wherein the regenerated captive target compound is supplied to the reservoir as a stripping agent at least partly to strip out therefrom any residual active agent.

23. (canceled)

24. The process according to claim 1 wherein the liquid mixture or solution that is sparged at least partially through the enclosure contains the target compound and at least one other material and wherein the liquid mixture or solution is at least partially evaporated in the downdraft to recover, concentrate or crystallise the target compound.

25. The process according to claim 24 wherein the target compound is a crystallisable or dryable material and crystallizes or dries in the enclosure on evaporation of the liquid mixture or solution.

26. The process according to claim 24 wherein the target compound is recovered as a concentrated stream following evaporation of the liquid solution or mixture.

27. The process according to claim 1 wherein at least part of the gaseous and/or vaporous mixture that is vented from the enclosure is directed to pass through a flowing stripping medium in order to recover any extraneous active agent or target compound from the vented gaseous and/or vaporous mixture and wherein the flowing stripping medium comprises a water curtain.

28. (canceled)

29. The process according to claim 1 wherein louvers are provided for directing gaseous and/or vaporous mixture downwardly into the enclosure and wherein the sparging device is arranged to distribute the liquid mixture or solution across at least a major part of a cross-sectional area of the enclosure, such that falling sparged liquid mixture or solution creates a downdraft in the enclosure.

30. (canceled)

31. The process according to claim 1 wherein the liquid mixture or solution has a vapour pressure such that at least partial evaporation of the liquid mixture or solution occurs in the enclosure and wherein the at least partial evaporation of the liquid mixture or solution in the enclosure accelerates the downdraft.

32-44. (canceled)

45. A capture tank for capturing a captive target compound from a gaseous and/or vaporous mixture comprising at least the captive target compound and one other material, or for recovering, concentrating or crystallising a target compound from a liquid mixture or solution comprising the target compound and at least one other material, the capture tank comprising:
an enclosure having a top region, a bottom region and at least one side defining the enclosure, the enclosure being at least partly open in its top region in order to communicate in use of the capture tank with a gaseous and/or vaporous mixture and for permitting ingress of the gaseous and/or vaporous mixture into the enclosure;
the enclosure communicating in its bottom region with a reservoir for receiving the captured captive target compound; having an opening associated with its at least one side and/or its bottom region for permitting egress from the enclosure of the gaseous and/or vaporous mixture optionally in at least partially captive target compound-depleted form; and
a sparging device for sparging at least partially through the enclosure from top to bottom a liquid mixture or solution for entraining the gaseous and/or vaporous mixture in the enclosure and carrying the entrained gaseous and/or vaporous mixture towards the bottom region of the enclosure.

46. (canceled)

47. The capture tank according to claim 45 wherein the capture tank is provided with a fill material selected from the group consisting of: a fill pack, an open cell structure, a foam, or another compressible to facilitate transport and storage.

48-51. (canceled)

52. The capture tank according to claim 45 wherein the at least one side wall has a cross sectional shape selected from the group consisting of: circular, ovoid, polygonal or irregular; and wherein the cross sectional area of the enclosure is at least about 1 m², or at least about 5 m², or at least about 10 m², or at least about 50 m², or at least about 100 m², or at least about 250 m², or at least about 500 m².

53. The capture tank according to claim 52 wherein the at least one side wall is constructed from the group consisting of: a flexible material, a solid material, blocks, bricks and panels.

54. A capture tank according to claim 45 wherein the opening for permitting egress of the gaseous and/or vaporous compound comprises one or more vents in the at least one side wall, wherein the one or more vents is or are provided towards or in the bottom region of the enclosure, and wherein the sparging device is situated towards the top region of the enclosure.
